(19) United States
(12) Patent Application Publication
(10) Pub. No.: US 2007/0117203 A1
(43) Pub. Date: May 24, 2007

(54) Modulation of stem and progenitor cell growth by oscillatory fluid flow

(76) Inventors: Christopher R. Jacobs, San Carlos, CA (US); Ying Jun Li, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/454,190

(22) Filed: Jun. 14, 2006

Related U.S. Application Data
(60) Provisional application No. 60/690,700, filed on Jun. 14, 2005.

(51) Int. Cl. C12N 5/08; C12N 5/06

(52) U.S. Cl. 435/325; 435/372

(57) ABSTRACT

Stem or progenitor cells, including cells derived from bone marrow, exhibit increased intracellular Ca²⁺ mobilization and alterations in gene expression and proliferation when subjected to an oscillatory fluid flow.

This invention was made with Government support under contracts AR45989 awarded by the National Institutes of Health and Government support under contract DAMD 17-98-1-8509 awarded by the Department of the Army. The Government has certain rights in this invention.

### BACKGROUND OF THE INVENTION

The regulation of progenitor and stem cell growth and differentiation is of great interest for regenerative medicine. Stem cells are unspecialized cells that can renew themselves indefinitely and develop into more mature, specialized cells. They are found in embryos during early stages of development, in fetal tissue, and more rarely, in some adult organs.

It is hoped that stem cells can be used to generate specific tissues, such as heart, lung, or kidney tissue, which could then help repair damaged and diseased organs or provide alternatives to organ transplants. Many of the diseases cited as targets for stem cell therapy, such as diabetes, heart disease, spinal cord injury, and Parkinson's disease, have few or no alternative treatment options.

Among adult tissues, bone marrow is a particularly rich source in stem and progenitor cells. The bone marrow (BM) is composed of the non-adherent hematopoietic and adherent stromal cell compartment. This adherent BM stromal cell fraction contains pluripotent mesenchymal stem cells (MSCs) and differentiated mesenchymal BM stromal cells. The MSCs self-renew by proliferation while maintaining their stem-cell phenotype and give rise to the differentiated stromal cells which belong to the osteogenic, chondrogenic, adipogenic, myogenic and fibroblastic lineages. A more primitive adherent stem cell was recently identified, the multipotent adult progenitor cell (MAPC) or mesodermal progenitor cell, which co-purifies with MSCs. These MAPCs differentiate into MSCs, endothelial, epithelial and even hematopoietic cells. BM stroma and hematopoietic cells are attractive targets for regenerative medicine.

Mechanical loading plays an important role in regulating bone metabolism. Increased mechanical loading increases bone formation and decreases bone resorption. The absence of mechanical stimulation causes reduced bone matrix protein production, mineral content, and bone formation, as well as an increase in bone resorption. However, the mechanism by which bone cells sense and respond to their physical environment is still poorly understood.

One of the important mediators cellular biophysical signals of mechanical loading is fluid flow. Oscillatory fluid flow, which occurs in vivo because of mechanical loading, was demonstrated to be a potentially important physical signal for loading-induced changes in bone cell metabolism. Relative to other loading-induced biophysical signals applied to cells in vitro, fluid flow appears to be significantly more potent at physiological levels.

The origin of loading-induced fluid flow is a consequence of the fact that a significant component of bone tissue is unbound fluid. Bone tissue contains an extracellular fluid compartment that has been demonstrated to communicate with the vascular compartment, and mechanical loading has been shown to enhance fluid exchange between the two spaces.

When bone is exposed to mechanical loading fluid in the matrix is pressurized and tends to flow into haversian canals. As loading is removed (e.g. during the gait cycle) the pressure gradients, and consequently the direction of fluid flow, are reversed resulting in a flow-time history experienced by the cells that is oscillatory in nature.

The modulation of stem and progenitor cell growth and differentiation is of great interest for clinical and research purposes. The role of oscillatory fluid flow in regulating these cells is described herein.

### SUMMARY OF THE INVENTION

Methods are provided for the modulating the growth and differentiation of progenitor or stem cells in vitro. Cells of particular interest include cells of the bone marrow, which include mesenchymal stem cells, hematopoietic stem cells, hematopoietic progenitor cells, osteoprogenitors, and the like. By providing for oscillatory fluid flow (OFF) in culture conditions, specific patterns of gene expression are induced. Progenitor cells have also been found to proliferate in response to OFF. The resulting cell populations are useful as a source of stem and progenitor cells, e.g. to reconstitute function in a host that is deficient in a particular cell lineage or lineages. The cells are also useful as a source of materials such as mRNA; for studying the effects of candidate pharmacologic agents, and the like.

### BRIEF DESCRIPTION OF THE DRAWINGS

FIG. 1. Example Ca²⁺ time history traces where each trace represents [Ca²⁺]ᵢ levels in one cell. Cells responded to OFF with transient increases in [Ca²⁺]ᵢ.

FIGS. 2A-2B. A. Percent of cells responding to OFF: A cell's responsiveness was quantified as exhibiting increased [Ca²⁺]ᵢ levels in response to flow of more than four times its pre-flow variability. The control group is the % of cells that responded during the pre-flow period. + indicates a significant difference from control (p<0.0001). B. Mean change in [Ca²⁺]ᵢ of cells (of the ones that responded): + indicates significance difference between OFF and control (p<0.001).

FIG. 3. OFF increases BrdU incorporation for MSCs. Cells exhibiting BrdU incorporated 24 hours after OFF. + indicates significant difference between MSCs exposed to OFF (n=15) and MSCs not exposed to OFF (n=25) (p<0.05).

FIG. 4. Effects of OFF on relative mRNA levels for MSCs: mRNA isolation 24 hours after OFF. + indicates significant difference between MSCs subjected to OFF and MSCs not exposed to OFF (p<0.05; n=17-22).

FIG. 5. Alkaline phosphatase levels of MSCs cultured in osteogenic media. ALP activity assayed 3 days after OFF. + indicates significant difference between MSCs subjected to OFF and MSCs not subjected to OFF (p<0.05, n

FIG. 6: An example of typical MSCs response without pharmacological agents. Each trace is one cell's intracellular calcium concentration.

FIG. 7: The percent of cells responding to oscillatory fluid flow in the presence of different calcium channel blockers.

### DESCRIPTION OF THE SPECIFIC EMBODIMENTS

Mammalian progenitor or stem cells are cultured in vitro in the presence of OFF. Cells of particular interest include cells of the bone marrow. By providing for oscillatory fluid flow (OFF) in culture conditions, specific patterns of gene expression are induced. Progenitor cells have also been found to proliferate in response to OFF. The resulting cell populations are useful as a source of stem and progenitor cells, e.g. to reconstitute function in a host that is deficient in a particular cell lineage or lineages. The cells are also useful as a source of materials such as mRNA; for studying the effects of candidate pharmacologic agents, and the like.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Stem cell: The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

Stem cells of interest include hematopoietic stem cells and progenitor cells derived therefrom (U.S. Pat. No. 5,061,620); neural crest stem cells (see Morrison et al. (1999) Cell 96:737-749); embryonic stem cells; mesenchymal stem cells; mesodermal stem cells; etc.

Other hematopoietic “progenitor” cells of interest include cells dedicated to lymphoid lineages, e.g. immature T cell and B cell populations. The methods of the present invention are useful in expanding selected populations of these cells.

Purified populations of stem or progenitor cells may be used to initiate the cultures. For example, human hematopoietic stem cells may be positively selected using antibodies specific for CD34, thy-1; or negatively selected using lineage specific markers which may include glycophorin A, CD3, CD24, CD16, CD14, CD38, CD45RA, CD36, CD2, CD19, CD56, CD66a, and CD66b; T cell specific markers, tumor specific markers, etc. Markers useful for the separation of mesodermal stem cells include FcγRII, FcγRIII, Thy-1, CD44, VLA-4α, LFA-1β, HSA, ICAM-1, CD45, Aa4.1, Sca-1, etc. Neural crest stem cells may be positively selected with antibodies specific for low-affinity nerve growth factor receptor (LNGFR), and negatively selected for the markers sulfatide, glial fibrillary acidic protein (GFAP), myelin protein Pₒ, peripherin and neurofilament. Human mesenchymal stem cells may be positively separated using the markers SH2, SH3 and SH4.

The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

The cells which are employed may be fresh, frozen, or have been subject to prior culture. They may be fetal, neonate, adult. Hematopoietic cells may be obtained from fetal liver, bone marrow, blood, particularly G-CSF or GM-CSF mobilized peripheral blood, or any other conventional source. The manner in which the stem cells are separated from other cells of the hematopoietic or other lineage is not critical to this invention. As described above, a substantially homogeneous population of stem or progenitor cells may be obtained by selective isolation of cells free of markers associated with differentiated cells, while displaying epitopic characteristics associated with the stem cells.

Oscillatory Fluid Flow. As used herein, OFF refers to conditions in which there is a reversal in flow experienced by the cells that is oscillatory in nature. An apparatus for such conditions is described, for example, by Jacobs et al. (1998) J. Biomechanics 31(11):969-97, herein specifically incorporated by reference, although it will be understood by one of skill in the art that various configurations are readily designed for this purpose. One in vitro culture system consists of a parallel plate flow chamber with a glass slide covered with cultured cells making up one side of the chamber. Fluid is delivered to the chamber via glass syringes mounted in a computer controlled materials testing device. Thus a wide variety of flow profiles can be programmed and delivered to the chambers within a frequency range from 0 to 30 Hz. An exemplary baseline flow exposure profile is a sinusoidal velocity profile designed to result in a peak wall shear stress of 2 Pa at a frequency of 2 Hz. In some circumstances, OFF may be induced in vivo.

Culture medium: The stem or progenitor cells are grown in vitro in an appropriate liquid nutrient medium. Generally, the seeding level will be at least about 10 cells/ml, more usually at least about 100 cells/ml and generally not more than about 10⁵ cells/ml, usually not more than about 10⁴ cells/ml.

Various media are commercially available and may be used, including Ex vivo serum free medium; Dulbecco's Modified Eagle Medium (DMEM), RPMI, Iscove's medium, etc. The medium may be supplemented with serum or with defined additives. Appropriate antibiotics to prevent bacterial growth and other additives, such as pyruvate (0.1-5 mM), glutamine (0.5-5 mM), 2-mercaptoethanol (1-10×10⁻⁵ M) may also be included.

### Cells

A population of cells comprising progenitor and/or stem cells is cultured in vitro in the presence of OFF, as described above. The population may be a complex population comprising a minority of progenitor and/or stem cells, or may be a purified population of stem and/or progenitor cells. The presence of OFF induces proliferation of the progenitor and/or stem cells, as well as inducing specific gene expression profiles. The number of assayable progenitor cells may be demonstrated by a number of assays. After one week the progenitor cell cloning efficiency will usually be at least about 75% that of the starting cell population, more usually 100% that of the starting cell population, and may be as high as 200% that of the starting cell population.

Frequently stem cells are isolated from biological sources in a quiescent state. After seeding the culture medium, the culture medium is maintained under conventional conditions for growth of mammalian cells, generally about 37° C. and 5% CO₂ in 100% humidified atmosphere. Fresh media may be conveniently replaced, in part, by removing a portion of the media and replacing it with fresh media. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

These cells may find various applications for a wide variety of purposes. The cell populations may be used for screening various additives for their effect on growth and the mature differentiation of the cells. In this manner, compounds which are complementary, agonistic, antagonistic or inactive may be screened, determining the effect of the compound in relationship with one or more of the different cytokines.

The populations may be employed as grafts for transplantation. For example, hematopoietic cells are used to treat malignancies, bone marrow failure states and congenital metabolic, immunologic and hematologic disorders. Marrow samples may be taken from patients with cancer, and enriched populations of hematopoietic stem cells isolated by means of density centrifugation, counterflow centrifugal elutriation, monoclonal antibody labeling and fluorescence activated cell sorting. The stem cells in this cell population are then expanded in vitro and can serve as a graft for autologous marrow transplantation. The graft will be infused after the patient has received curative chemo-radiotherapy.

### Libraries

The cells of this invention can be used to prepare a cDNA library relatively uncontaminated with cDNA preferentially expressed in cells from other lineages. For example, progenitor/stem cells are cultured in the presence of OFF, and then mRNA is prepared from the pellet by standard techniques (Sambrook et al., supra). After reverse transcribing into cDNA, the preparation can be subtracted with cDNA from other progenitor cells, or end-stage cells from the relevant developmental pathway.

Of particular interest is the examination of gene expression in the stem/progenitor cells of the invention. The expressed set of genes may be compared against other subsets of cells, against other stem or progenitor cells, against adult muscle tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of polypeptide of interest. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

### Screening Assays

The cells are also useful for in vitro assays and screening to detect factors that are active on stem/progenitor cells. Of particular interest are screening assays for agents that are active on human cells. A wide variety of assays may be used for this purpose, including immunoassays for protein binding; determination of cell growth, differentiation and functional activity; production of factors; and the like.

In screening assays for biologically active agents, viruses, etc. the subject cells, usually a culture comprising the subject cells, is contacted with the agent of interest, and the effect of the agent assessed by monitoring output parameters, such as expression of markers, cell viability, and the like. The cells may be freshly isolated, cultured, genetically altered as described above, or the like. The cells may be environmentally induced variants of clonal cultures: e.g. split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Experimental

### EXAMPLE 1

### Effects of Loading Induced Oscillatory Fluid Flow on Human Marrow Stromal Cell Proliferation and Differentiation

Oscillatory fluid flow (OFF) is hypothesized to be an important regulator of progenitor cells, including osteoprogenitors. Mechanical loading induces fluid flow in bone tissue and this flow has been found to be a potent regulator of osteoblastic and osteocytic cells. In our previous studies of osteoblastic (MC3T3-E1, hFOB) and osteocytic (MLO-Y4) cells, we found the effects of OFF to include intracellular Ca²⁺ mobilization, prostaglandin E₂ (PGE₂) release, increased osteopontin (OP) gene expression, increased mitogen-activated protein (MAP) kinase activity, and inhibition of NF-κB-DNA binding activities. The effect on progenitor cells, such as marrow stromal cells, is unknown.

Marrow stromal cells (MSCs) were chosen for this study based on the substantial evidence that they contain osteoprogenitors. Through selective culturing techniques that isolate progenitor cells from other marrow cells, MSCs can now be enriched considerably from bone marrow aspirates. In vivo, MSCs were isolated from donor rats, loaded into transplantation vehicles, and placed subcutaneously in recipient rats. Subsequently, the donor cells in the transplantation vehicles formed mineralized bone. In addition, when transplanted into the marrow cavity of the recipient, donor MSCs migrated throughout the body and became long-term precursors for bone tissue. Additionally, in vitro studies have shown that biochemical signals can be added to MSC cultures to regulate the differentiation of the cells down the osteoblast, chondrocyte or adipocyte lineages.

The purpose of this study was to examine the effects of OFF on MSCs. The experiments consisted of quantifying intracellular Ca²⁺ mobilization, cell proliferation, gene expression, and alkaline phosphatase (ALP) activity. Ca²⁺ is an important signaling molecule and has been shown to have an important role in the adaptation of bone to mechanical loading. Additionally, we quantified the proliferation rate of MSCs exposed to OFF because MSC proliferation rate directly affects the number of osteoprogenitors available for recruitment into osteoblasts. Furthermore, MSC differentiation was studied by considering gene expression and ALP activity. The mRNA levels of four osteoblast marker genes were examined: core binding factor 1 (Cbfa1), osteopontin (OP), osteocalcin (OSTC), and type I collagen (Col I). Previous studies indicate that Cbfa1 is a necessary transcriptional activator of osteoblast differentiation and is localized to the skeletal system. OP is a mineral-binding, non-collagenous, matrix protein and is thought to promote osteoblast attachment and migration. OSTC is a non-collagenous protein found exclusively in the bone extracellular matrix and is thought to be upregulated at the onset of mineralization. Col I is the most abundant extracellular protein in bone and is expressed during all stages of osteoblast development. Finally, ALP activity is required for bone matrix mineralization and is used to assess osteoblast activity. Previous studies have also found ALP activity to be a marker of the various stages of osteoblast differentiation, including the differentiation of MSCs into osteoblast-like cells. By examining intracellular Ca²⁺ mobilization, cell proliferation, gene expression, and ALP activity, this study advances the understanding of mechanical regulation of MSCs.

Materials and Methods:

Experimental Design: The effects of OFF on MSCs were studied using Ca²⁺ mobilization, cell proliferation, gene expression, and ALP activity. The Ca²⁺ experiment was used to determine if OFF can produce an intracellular [Ca²⁺] increase. The effect of OFF on MSC proliferation was studied because MSC proliferation directly affects the number of available osteoprogenitors. Hence, cells cultured subjected to OFF were compared to cells not subjected to OFF. The gene expression experiment was conducted to determine whether the mechanical signal OFF alone could stimulate MSC differentiation. Finally, the ALP activity experiment was conducted to determine whether OFF modulated the osteogenic differentiation of MSCs. MSCs were first cultured in media which stimulated osteogenesis so that ALP activity was at a measurable level, and then the cells were subjected to OFF.

Materials: Reagents were purchased from the following companies: BioWhittaker (Walkersville, Md.)—cryopreserved human mesenchymal stem cells (MSCs), Dulbecco's Modified Eagle Media (DMEM), Fetal Bovine Serum (FBS), L-Glutamine. Gibco/Life Technologies (Rockville, Md.)—α-MEM, Penicillin/Streptomycin, Trypsin/EDTA, Phosphate Buffered Saline (PBS). Sigma (St. Louis, Mo.)—dexamethasone, β-glycerophosphate, poly-I-lysine, alkaline phosphatase kit 104. Qiagen (Valencia, Calif.)—RNeasy Mini Kit. Wako Chemicals (Richmond, Va.)—ascorbic acid-2 phosphate. Molecular Probes (Eugene, Oreg.)—Fura-2 AM, Fura-2 AM calibration kit, Absolute-S SBIP Cell Proliferation Assay Kit (A-23150). Pierce (Rockford, Ill.)—BCA Protein Assay Kit.

Cell Culture: MSCs were purchased from BioWhittaker. At BioWhittaker, MSCs were obtained from healthy human volunteers by bone marrow aspiration followed by density gradient centrifugation and selective culturing techniques. These cells have been shown to be mesenchymal progenitors and to be able to differentiate into osteoblastic cells. At our lab, cells were thawed and cultured at standard conditions of 37° C., 5% CO₂, and 95% humidity. Growth media consisted of DMEM, 10% FBS, 2% L-Glutamine, and 1% penicillin/streptomycin. Fresh media was added every 3-4 days, and cells were subcultured every 7 days using 0.05% Trypsin-0.53 mM EDTA.

Oscillatory Fluid Flow (OFF): Cells were subjected to OFF in a parallel plate flow chamber, as described by Jacobs et al. (1998) J Biomech 31:969-76, herein specifically incorporated by reference with respect to such methods and apparatus. Briefly, cells were cultured on slides and placed against a polycarbonate manifold, where a gasket maintained a uniform gap between the two parallel plates. The flow channel geometry was 38×10×0.28 mm for the calcium imaging experiments and 60×24×0.28 mm for all other experiments. Since the Reynolds number for the flow rate used was below 50 and well within the laminar flow region, the shear stress on the cells could be calculated using Poiseuille's equation. The chamber was sealed and fluid flow was delivered to the inlet by gastight Hamilton syringes mounted between electromechanical actuators (ElectroForce Actuator, EnduraTec, Minnetonka, Minn.). The actuators were controlled for maximum displacement, waveform, and frequency using Wintest software (EnduraTec). Flow parameters were verified using a high-frequency ultrasonic flow probe (Transonic Systems Inc., Ithaca, N.Y.).

Calcium Imaging: Intracellular levels of Ca²⁺ were quantified using a ratiometric imaging technique with the Ca²⁺ sensitive dye Fura-2 Acetoxymethyl ester (Fura-2 AM). Fura-2 AM exhibits a shift in absorption when illuminated with UV light such that its emission intensity increases with Ca²⁺ concentration when excited at 340 nm and decreases with Ca²⁺ concentration when excited at 380 nm. The ratio of the emission intensities F₃₄₀/F₃₈₀ is a quantitative measurement of intracellular calcium [Ca²⁺]ᵢ.

Cells were cultured on UV transparent quartz slides for 3 days. Prior to exposure to OFF, cells were washed with PBS, incubated with 10 μM Fura-2 AM in A-MEM and 2% FBS for 30 min at 37° C., and then washed again with PBS. Following Fura-2 loading, the slides were mounted in a parallel plate flow chamber, and then fixed to the stage of a Nikon TE300 epi-fluorescent microscope (Technical Instruments SF, Burlingame, Calif.). The setup was left undisturbed for 30 min, during which the cells were perfused with media at 0.05 ml/min. Cells were then subjected to 3 minutes of OFF at 1 Hz with peak shear stress of 20 dynes/cm². Ratio images were acquired every 2 seconds for 3 minutes prior to flow, the duration of the OFF, and for 3 minutes after the flow was terminated. Ratio images were recorded and analyzed using image analysis software (Metafluor; Universal Imaging, West Chester, Pa.). Fura-2 ratio values were converted to [Ca²⁺]ᵢ levels with a calibration curve derived from a series of standard Ca²⁺ solutions provided by the manufacturer.

Cell proliferation: Cell proliferation was assessed by examining the incorporation of 5-bromo-2′-deoxyuridine (BrdU), a thymidine analog (Absolute-S SBIP Cell Proliferation Assay Kit). For the BrdU, gene expression and ALP activity experiments, cells were cultured on 0.01% poly-l-lysine coated glass slides for 3-5 days, then loaded onto the flow chambers and subjected to OFF for 2 hours at 1 Hz with peak shear stress of 10 dynes/cm² and perfused with fresh media at 0.05 ml/min. The no flow (control) cells remained in the incubator. Cells were collected 24 hours after OFF and incubated with BrdU for 30 minutes. Then the cells were photolysed and labeled. BrdU was detected by an anti-BrdU antibody using standard immunohistochemical techniques. The fraction of BrdU-positive cells was counted using the epi-fluorescent microscope.

Gene expression: Gene expression was quantified using real time reverse transcriptase polymerase chain reaction (real time RT-PCR). Four osteoblast marker genes were studied: Cbfa1, OP, OSTC, and Col I. Cells were cultured on coated glass slides for 3-5 days and subjected to OFF, as described above. 24 hours after OFF, total RNA was isolated using the RNeasy kit. Cells were lysed, homogenized, and bound to a silica-gel-based membrane to obtain high quality total RNA. Real time RT-PCR was performed at the Penn State Nucleic Acid Facility using the ABI PRISM 7700 Sequence Detection system (Applied Biosystems, Foster City, Calif.). For each sample, mRNA levels for each gene were first normalized to the 18S rRNA levels, and then normalized to the negative control.

The following primers and probes were used: Cbfa1: the forward primer was 5′FAM-TGC TTC ATT CGC CTC ACA M-TAMRA-3′; the reverse primer was 5′FAM-TGC TGT CCT CCT CGA GM AGT T-TAMRA-3′; and the probe was 5′-MC CAC AGA ACC ACA AGT GCG GTG C-3′. OP: the forward primer was 5′FAM-TTG CAG CCT TCT CAG CCA A-TAMRA-3′; the reverse primer was 5′FAM-CM MG CM ATC ACT GCA ATT CTC-TAMRA-3′; and the probe was 5′-CGC GGA CCA AGG MA ACT CAC TAC CA-3′. Col I: the forward primer was 5′FAM-CGC ACG GCC MG AGG A-TAMRA-3′; the reverse primer was 5′FAM-ACG CAG GTG ATT GGT GGG-TAMRA-3′; and the probe was 5′-CM GTC GAG GGC CM GAC GM GAC A-3′. OSTC: the forward primer was 5′FAM-GCA GGT GCG MG CCC A-TAMRA-3′; the reverse primer was 5′FAM-ACC CTA GAC CGG GCC GT-TAMRA-3′; and the probe was 5′-TTT CAG GAG GCC TAT CGG CGC TTC-3′.

Alkaline phosphatase: ALP activity was measured by the conversion of p-nitrophenyl phosphate to p-nitrophenol (Sigma 104 kit). Cells were cultured on coated glass slides for 3-5 days and subjected to OFF, as described above. For the ALP activity experiments, cells were exposed to osteogenic supplements: 5 nM dexamethasone, 0.025 mM ascorbic acid-2 phosphate, and 5 mM β-glycerophosphate. Without these supplements, ALP activity was too low to be accurately measured (data not shown). ALP activity was assayed 3 days after OFF, since previous studies found maximum ALP activity at days 6-8. Cells were lysed using 0.05% triton and freeze-thawed twice, then the substrate was added. The end-product, p-nitrophenol, was measured by absorption at 415 nm and converted to Sigma units using a p-nitrophenol standard absorption curve. In parallel, protein concentration from each slide was measured to normalize ALP activity by cell count (Pierce BCA kit).

Data analysis: Calcium imaging data was analyzed using the Rainflow cycle counting numerical procedure. This method identifies oscillations in the time history data and is used to quantify [Ca²⁺]ᵢ responses from background noise. Calcium imaging data is expressed as the fraction of cells responding i SE and mean change in [Ca²⁺]ᵢ±SE. Statistical analysis using one-way ANOVA and Fisher's Protected Least Significant Difference was utilized to detect significant differences between groups; p<0.05 was considered statistically significant.

BrdU incorporation, gene expression, and ALP activity data were expressed as means±SE. Statistical analyses were performed using two-way ANOVA followed by the Dunnett's post hoc test using StatView software (Cary, N.C.); p<0.05 was considered statistically significant.

Results:

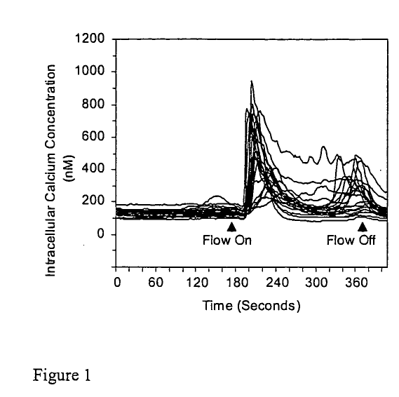

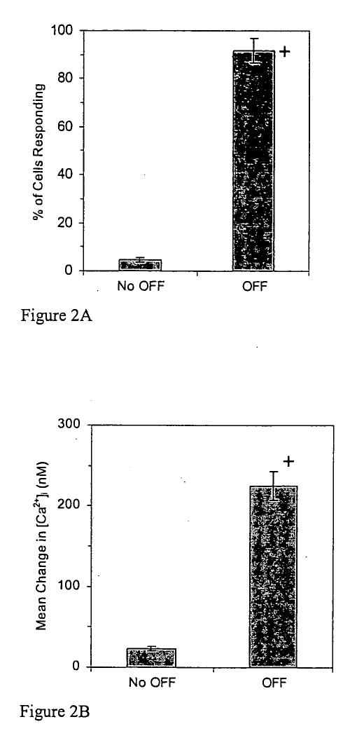

Calcium Imaging: OFF triggered potent [Ca²⁺]ᵢ responses in MSCs. Two parameters were used to quantify the responsiveness: the fraction of cells that respond to OFF and the mean change in [Ca²⁺]ᵢ amplitude. A responding cell was defined as one exhibiting increased intracellular calcium levels in response to flow of more than four times its maximum pre-flow variability, as described previously. 3 slides were observed for a total of 60 cells. FIG. 1 shows examples of [Ca²⁺]ᵢ time history traces, where each trace represents [Ca²⁺]ᵢ levels in one cell. When OFF was applied, one or more dramatic and transient increases in [Ca²⁺]ᵢ were observed. FIG. 2A shows the fraction of cells that responded to OFF (89±5.4% of cells) was significantly greater than the pre-flow control period (4.6±2.5%, p<0.0001). FIG. 2B shows that of the cells with [Ca²⁺]ᵢ increases, the mean change in [Ca²⁺]ᵢ was significantly higher during OFF (224 nM±129, p<0.001) than during the pre-flow control period (23 nM±27, p<0.001).

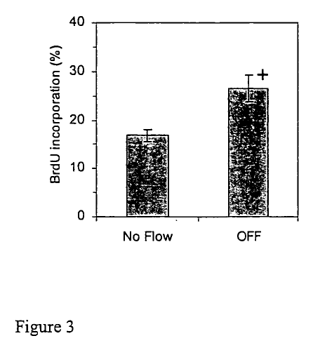

Cell Proliferation: Cell proliferation experiments revealed that OFF significantly increased the proliferation of MSCs. FIG. 3 shows that MSCs subjected to OFF had significantly higher fraction of BrdU incorporation than MSCs not subjected to OFF (57% increase, n=15 for cells exposed to OFF, n=25 for cells not exposed to OFF).

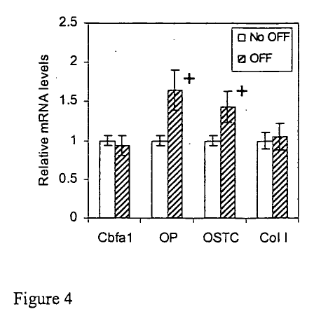

Gene expression: OFF significantly altered the gene expression of MSCs. Six experiments were conducted with total sample size (n) of 17 to 22 for each experimental group. FIG. 4A shows that cells subjected to OFF had significantly higher OP (65% increase) and OSTC (44% increase) mRNA levels. The Cbfa1 and Col I mRNA levels were not significantly different.

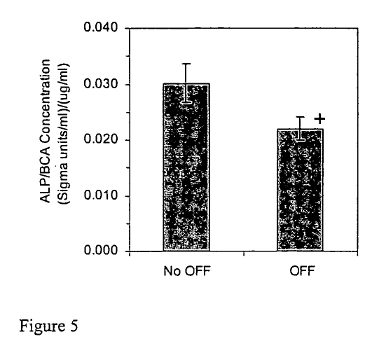

Alkaline Phosphatase: OFF significantly decreased ALP activity in MSCs cultured in osteogenic media. ALP activity was measured by the accumulation of the reaction product p-nitrophenol after 5, 10, 20, and 30 minutes of incubation. For all samples, p-nitrophenol levels increased over time and were not saturated at 30 minutes. FIG. 5 shows the 30 min reaction product level normalized for protein concentration. MSCs subjected to OFF had significantly lower ALP activity than MSCs not subjected to flow (27% decrease, n=9).

The results of this study show that MSCs respond to OFF with transient increases in intracellular Ca²⁺, increased proliferation rate, upregulation in osteoblastic gene expression, and decreased ALP activity. This study also demonstrated that OFF increased the proliferation of MSCs. There is an important link between osteoprogenitor proliferation and bone remodeling because osteoblasts are constantly renewed by osteoprogenitors in healthy bone. The MSCs used in the study were a heterogeneous mixture containing both stem cells and progenitor cells.

The gene expression results suggest that OFF affects the osteogenic differentiation of MSCs. Cbfa1, a mammalian homolog of Drosophilia Runt protein, is a critical regulator of osteoblast differentiation. Cbfa1 binds to the OSE2, a cis acting element in the promoter of OSTC and has affinity to promoters of α1 collagen and OP. Genetically altered Cbfa1-deficient mice have a skeleton devoid of osteoblasts and bone matrix. Our finding that OFF upregulated OP and OSTC but did not upregulate Cbfa1 suggests that OFF may act downstream of Cbfa1 to stimulate OP and OSTC. For example, osterix (Osx) is a transcription factor that regulates osteoblastic differentiation downstream of Cbfa1. Osx-deficient mice lack osteoblasts and bone matrix even though Cbfa1 is expressed normally.

OP and OSTC are indicators of osteoblastic differentiation, whose functions in the bone matrix continue to be intensely studied. Our results showed that OFF upregulated OP and OSTC, which suggests increased MSC osteoblastic differentiation. In bone, OP has been found to regulate bone cell adhesion, osteoclast function, and matrix mineralization. However, skeletal changes were not found in OP-null mice which indicate that other matrix molecules may substitute for OP. OSTC, a bone specific matrix protein, has increased expression during mineralization and is regulated by Cbfa1 and vitamin D₃. On the other hand, increased bone formation was found in OSTC-null mice. Mizuno et al found that OP and OSTC were upregulated in MSCs after mineralization (3 weeks culture time in a type I collagen matrix). In addition, intermittent mechanical strain has been found to upregulate OP level in the absence of hormonal Vitamin D stimulation.

OFF did not significantly altered Col I mRNA levels. Col I mRNA levels may not have been sensitive to these signals because they were already highly expressed in the MSCs. This finding is similar to Frank et al which found that MSCs cultured in osteogenic media did not show significant changes in Col I over a 20 day culture period (23).

ALP activity has been used in many studies as an indicator of osteoblast differentiation (41, 43, 52, 53, 55, 62, 72). We found that OFF inhibited ALP activity. This result may be interpreted in several ways. Several in vivo and in vitro studies have found ALP activity to increase after proliferation and to decrease immediately prior to mineralization (4, 37, 41, 55, 60, 64). Because OFF increased the proliferation of MSCs, this may delay their osteogenic differentiation since phenotypic changes often occur after proliferation stops. Stanford et al found that rat calvaria-derived osteoblast-like cells exhibited increased proliferation and depressed ALP activity as a response to mechanical deformation (71). A second possibility is that OFF may have been applied at a time point on the differentiation pathway when MSCs do not increase ALP activity in response to loading. For example, Thomas et al found that mechanical strain on MSCs decreased ALP activity if applied before day 8 in culture and increased ALP activity if applied later (72). In addition, Hillsley et al found that pulsatile fluid flow down-regulated ALP activity in osteoblasts (38). Yet another possibility is that OFF may have accelerated MSC osteogenic differentiation towards mineralization, since ALP activity has been found to decrease immediately prior to mineralization (41). A time course study examining ALP activity and mineralization is required to distinguish amongst these possibilities.

Insights into MSC response to mechanical signals will enable better understanding and treatment of bone loss diseases such as osteoporosis and disuse bone loss. While previous studies have focused on osteoblasts, osteocytes and osteoclasts, MSC response may contribute directly to bone's response to mechanical loading. In addition, MSCs offer novel treatment options in tissue engineering applications. Marrow cells can be harvested from an individual, then culture expanded in tissue scaffolds and subsequently re-implanted (10). The tissue scaffolds will need to both support cell growth and provide the biochemical and physical signals necessary for cell differentiation.

In summary, this study has found OFF to be a potent regulator of MSC proliferation and differentiation. OFF triggered intracellular Ca²⁺ mobilization, where Ca²⁺ is an important second messenger in the mechanotransduction pathway. OFF also significantly increased MSC proliferation, thereby increasing the number of potential osteoprogenitors. In addition, OFF upregulates osteoblastic gene expression of OP and OSTC in the absence of biochemical stimuli. Finally, OFF lowers the ALP activity of osteogenic MSC.

### EXAMPLE 2

### The Determination of the Mechanotransduction Pathway of Marrow Stromal Cells Exposed to Oscillatory Fluid Flow

The objectives of this study are to determine the short-term effects of Oscillatory Fluid Flow (OFF) on Marrow Stromal Cells (MSCs). When bone is exposed to mechanical loading, fluid in the matrix is pressurized and tends to flow into haversian canals. As loading is removed (e.g. during the gate cycle), the pressure gradients, and consequently the direction of the fluid flow, are reversed. This results in a flow-time history experienced by the MSCs that is oscillatory in nature.

Methods: This study investigated intracellular [Ca²⁺] as an osteogenic biological response variable. A variety of pharmacological agents were employed to attempt to block the calcium response to determine if OFF activated an intracellular or extracellular calcium response and if that response was transduced using stretch activated or voltage activated channels. A previously described technique was used to acquire the calcium data, at least 4 experiments (slides) were done for each experimental group.

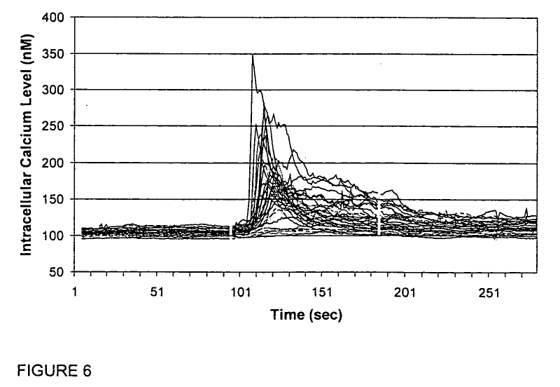

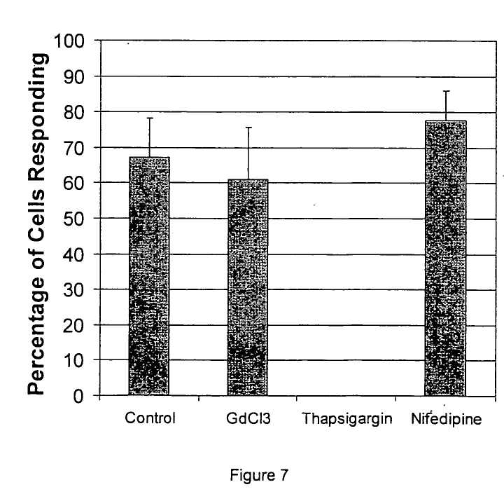

Ca²⁺ Responses to OFF—An example of typical MSCs response without pharmacological agents is shown in FIG. 6. Each trace is one cell's intracellular calcium concentration. A cell was classified as responding if the peak oscillation after the onset of flow was four times greater than that peak oscillation before the onset of flow. The percentage of cells that responded to OFF with a change in intracellular calcium was not different from control (67.1±11.2) in the presence of either a stretch activated channel blocker (gadolinium chloride (10 μM), 61.1±14.7) or a L-type voltage-operated calcium channel blocker (nifidipine (20 μM), 77.7±8.3). Thapsigargin (50 nM), which emptied the intracellular stores of calcium, caused a significant decrease (p<0.05) in cells responding to OFF completely eliminating the response (0±0). This data is shown in FIG. 7.

Our results suggest that the mechanisms for the calcium response of MSCs to OFF is the release of intracellular stores of calcium. This suggests a ubiquitous pathway in the transduction of the extracellular signal (OFF) into the cell interior.

What is claimed is:

1. A method of modulating the growth or differentiation of a stem or progenitor cell in vitro, the method comprising:
contacting said cell with an oscillatory fluid flow.

2. The method according to claim 1, wherein said cell is a bone marrow cell.

3. The method according to claim 2, wherein said bone marrow cell is a stromal cell.

4. The method according to claim 2, wherein said bone marrow cell is a hematopoietic stem or progenitor cell.

5. The method according to claim 2, wherein said bone marrow cell is a mesenchymal stem cell.

6. The method according to claim 3, wherein said cell is an osteoprogenitor cell.

7. An in vitro culture comprising:
a stem or progenitor cell in contact with an oscillatory fluid flow.

8. The method according to claim 7, wherein said cell is a bone marrow cell.

9. The method according to claim 8, wherein said bone marrow cell is a stromal cell.

10. The method according to claim 9, wherein said cell is an osteoprogenitor cell.

11. The method according to claim 8, wherein said bone marrow cell is a hematopoietic stem or progenitor cell.

12. The method according to claim 8, wherein said bone marrow cell is a mesenchymal stem cell.


SEQUENCE LISTING (12 sequences)

SEQ ID NO: 1 - DNA, 20 bp, Artificial Sequence
  synthetic primer
tgcttcattcgcctcacaaa

SEQ ID NO: 2 - DNA, 22 bp, Artificial Sequence
  synthetic primer
tgctgtcctcctcgagaaagtt

SEQ ID NO: 3 - DNA, 25 bp, Artificial Sequence
  synthetic primer
aaccacagaaccacaagtgcggtgc

SEQ ID NO: 4 - DNA, 19 bp, Artificial Sequence
  synthetic primer
ttgcagccttctcagccaa

SEQ ID NO: 5 - DNA, 24 bp, Artificial Sequence
  synthetic primer
caaaagcaaatcactgcaattctc

SEQ ID NO: 6 - DNA, 26 bp, Artificial Sequence
  synthetic primer
cgcggaccaaggaaaactcactacca

SEQ ID NO: 7 - DNA, 16 bp, Artificial Sequence
  synthetic primer
cgcacggccaagagga

SEQ ID NO: 8 - DNA, 18 bp, Artificial Sequence
  synthetic primer
acgcaggtgattggtggg

SEQ ID NO: 9 - DNA, 25 bp, Artificial Sequence
  synthetic primer
caagtcgagggccaagacgaagaca

SEQ ID NO: 10 - DNA, 16 bp, Artificial Sequence
  synthetic primer
gcaggtgcgaagccca

SEQ ID NO: 11 - DNA, 17 bp, Artificial Sequence
  synthetic primer
accctagaccgggccgt

SEQ ID NO: 12 - DNA, 24 bp, Artificial Sequence
  synthetic primer
tttcaggaggcctatcggcgcttc